(19) United States
(12) Patent Application Publication
(10) Pub. No.: US 2023/0167561 A1
(43) Pub. Date: Jun. 1, 2023

(54) Continuous Capture of Carbon Dioxide From Exhaust Gas and Conversion Thereof to Useful Chemistries

(71) Applicant: Halliburton Energy Services, Inc., Houston, TX (US)

(72) Inventors: Tatyana Vladimirov KHAMATNUROVA, Houston, TX (US); Philip D. NGUYEN, Houston, TX (US); Ronald Glen DUSTERHOFT, Houston, TX (US)

(21) Appl. No.: 17/533,478

(22) Filed: Nov. 23, 2021

(51) Int. Cl. C25B 3/07; B01J 23/46; C07C 29/154; C07C 29/156; C07C 29/152; C25B 3/26; C25B 11/02; C25B 11/091

(52) U.S. Cl. CPC C25B 3/07; B01J 23/462; C07C 29/152; C07C 29/154; C07C 29/156; C25B 3/26; C25B 11/02; C25B 11/091

(57) ABSTRACT

A method including collecting exhaust gas comprising carbon dioxide (CO₂) at a wellsite to provide a collected exhaust gas, separating CO₂ from the collected exhaust gas to provide a separated CO₂, and forming an alcohol product utilizing at least a portion of the separated CO₂. The alcohol product can include methanol, ethanol, a precursor thereof, or a combination thereof.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD

The present disclosure relates generally to systems and methods of sequestering carbon dioxide (CO₂). More specifically, this disclosure relates to collecting exhaust gas comprising CO₂ and forming an alcohol product utilizing at least a portion of the CO₂ in the collected exhaust gas. Still more specifically, this disclosure relates to collecting exhaust gas comprising CO₂, separating (e.g., high purity) CO₂ from the collected exhaust gas, and forming an alcohol product comprising methanol, ethanol, a precursor thereof, or a combination thereof utilizing at least a portion of the high purity CO₂.

### BACKGROUND

Natural resources (e.g., oil or gas) residing in a subterranean formation can be recovered by driving resources from the formation into a wellbore using, for example, a pressure gradient that exists between the formation and the wellbore, the force of gravity, displacement of the resources from the formation using a pump or the force of another fluid injected into the well or an adjacent well. A number of wellbore servicing fluids can be utilized during the formation and production from such wellbores. For example, in embodiments, the production of fluid in the formation can be increased by hydraulically fracturing the formation. That is, a treatment fluid (e.g., a fracturing fluid) can be pumped down the wellbore to the formation at a rate and a pressure sufficient to form fractures that extend into the formation, providing additional pathways through which the oil or gas can flow to the well. Subsequently, oil or gas residing in the subterranean formation can be recovered or “produced” from the well by driving the fluid into the well. During production of the oil or gas, substantial quantities of produced water, which can contain high levels of total dissolved solids (TDS), and produced gas can also be produced from the well, and a variety of exhaust gases and flare gases conventionally sent to flare can be formed For example, oil and gas wells produce oil, gas, and/or byproducts from subterranean formation hydrocarbon reservoirs. A variety of subterranean formation operations are utilized to obtain such hydrocarbons, such as drilling operations, completion operations, stimulation operations, production operations, enhanced recovery operations, and the like. Such subterranean formation operations typically use a large number of vehicles, heavy equipment, and other apparatus (collectively referred to as “machinery” herein) in order to achieve certain job requirements, such as treatment fluid pump rates. Such equipment may include, for example, pump trucks, sand trucks, cranes, conveyance equipment, mixing machinery, and the like. Many of these operations and machinery utilize combustion engines that produce exhaust gases (e.g., including carbon dioxide/greenhouse gas emissions) that are emitted into the atmosphere.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic flow diagram of a method, according to one or more embodiments of this disclosure,

FIG. 2 is a schematic of a system, according to one or more embodiments of the present disclosure;

FIG. 3 is a schematic of a plurality of machinery that may be located and operated a wellsite for performing a subterranean formation operation and may produce exhaust gas comprising CO₂, according to one or more embodiments of the present disclosure;

FIG. 4 is a schematic of an alcohol production apparatus, according to one or more embodiments of this disclosure, and

FIG. 5 is a high-resolution transmission electron microscopy of an electrocatalyst suitable for use in the system of FIG. 2, according to one or more embodiments of this disclosure.

### DETAILED DESCRIPTION

It should be understood at the outset that although an illustrative implementation of one or more embodiments are provided below, the disclosed systems and/or methods can be implemented using any number of techniques, whether currently known or in existence. The disclosure should in no way be limited to the illustrative implementations, drawings, and techniques below, including the exemplary designs and implementations illustrated and described herein, but can be modified within the scope of the appended claims along with their full scope of equivalents.

Carbon dioxide (CO₂) can be a main component of exhaust gas. Via this disclosure, exhaust gas and operational equipment emissions can be captured, CO₂ separated therefrom (e.g., adsorbed and desorbed), as needed, and the CO₂ sequestered. In embodiments, the systems and methods of this disclosure provide for capturing carbon dioxide (CO₂) emitted as part of the exhaust gas released at the wellsite, and utilizing the collected CO₂ to produce useful chemistries. In embodiments, the systems and methods disclosed herein enable value-added utilization of CO₂ from exhaust gas and conversion of the CO₂ to various alcohol products that can be utilized, for example, as-is or as feedstock for further processes, such as to produce polymers. For example, carbon dioxide can be hydrogenated with hydrogen to methanol. Methanol is a feedstock for multitude of chemicals and products and a superior fuel for internal combustion engines and fuel cells. Alcohol product comprising methanol, produced via the herein described carbon capture, can be utilized as is, converted to another product, or sold (e.g., as an additive or a raw material for feedstock). In embodiments, the disclosed system and method provide a portable and seamless process of making an alcohol product at a wellsite utilizing CO₂ from exhaust gas, whereby CO₂ emissions can be reduced.

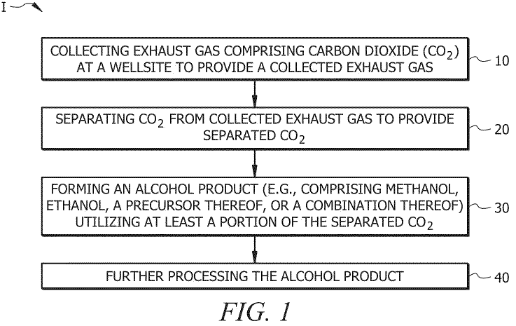

A method of this disclosure will now be described with reference to FIG. 1, which is a schematic flow diagram of a method I according to one or more embodiments of this disclosure. As seen in FIG. 1, method I includes collecting exhaust gas comprising carbon dioxide (CO₂) at a wellsite to provide a collected exhaust gas at 10, separating CO₂ from the collected exhaust gas to provide a separated CO₂ at 20, and forming an alcohol product utilizing at least a portion of the separated CO₂ at 30. The alcohol product can comprise methanol, ethanol, a precursor thereof (e.g., a precursor of methanol, a precursor of ethanol), or a combination thereof. As indicated in FIG. 1, a method I of this disclosure can further comprise further processing the alcohol product at 40. Further processing can include removing one or more components (e.g., byproducts, contaminants) from the alcohol product, and/or forming another product (eg., MTBE) from the alcohol product, as discussed further hereinbelow. Although depicted in a certain order in FIG. 1, in embodiments, one or more of steps 10 to 40 can be absent, and/or one or more of the steps 10 to 40 can be performed more than once and/or in a different order than described herein or depicted in the embodiment of FIG. 1. For example, separating step 20 and/or further processing step 40 can be absent, in some embodiments.

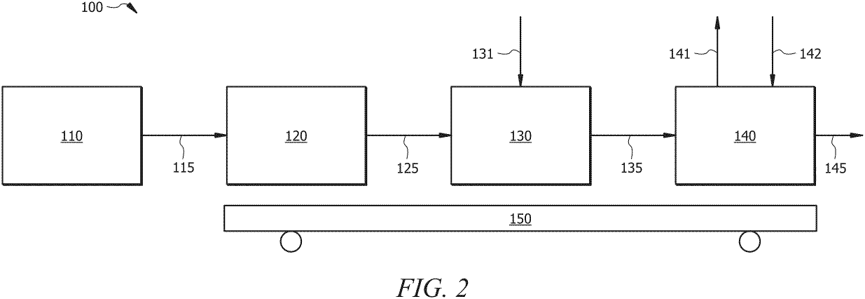

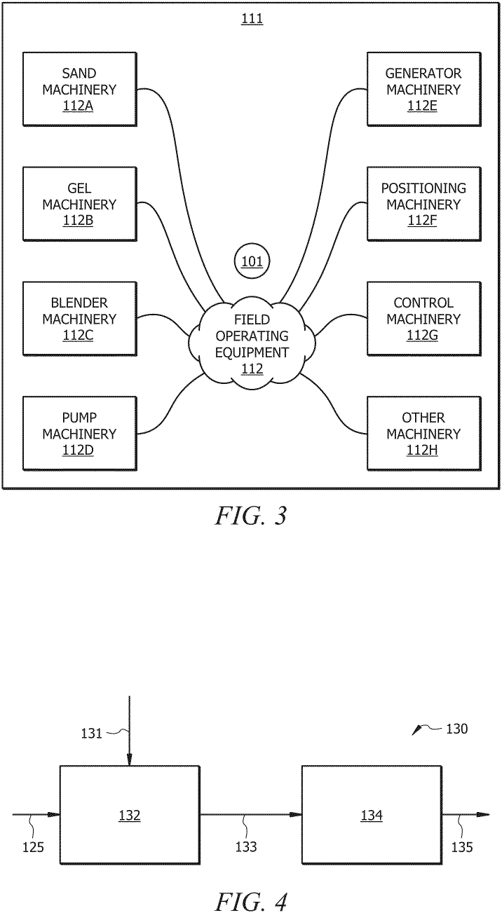

The method of this disclosure will now be detailed and a system for carrying out the method according to one or more embodiments of this disclosure described with reference to FIG. 2, which is a schematic of a system 100 according to one or more embodiments of this disclosure, FIG. 3, which is a schematic of a plurality of machinery that may be located and operated at a wellsite for performing a subterranean formation operation and may produce exhaust gas comprising CO₂, according to one or more embodiments of the present disclosure, and FIG. 4, which is a schematic of an example alcohol production apparatus 130 that can be utilized in a system 100, in one or more embodiments.

With reference now to FIG. 2, system 100 comprises: an exhaust gas collection system 110 configured for collecting exhaust gas 115 comprising carbon dioxide (CO₂) at a wellsite 111 (FIG. 3) to provide a collected exhaust gas (e.g., step 10 of FIG. 1); a CO₂ separation apparatus 120 configured for separating CO₂ from the collected exhaust gas 115 to provide a separated CO₂ 125 (e.g., step 20 of FIG. 1); and an alcohol production apparatus 130 configured for forming an alcohol product or precursor 135 (referred to herein for simplicity as “alcohol product” 135) utilizing at least a portion of the separated CO₂ 125 (e.g., step 30 of FIG. 1). The alcohol product comprises one or more alcohols, such as methanol, and/or ethanol, a precursor of one or more alcohol (eg., a precursor to methanol, a precursor to ethanol), or a combination thereof. As depicted in FIG. 2, system 100 can further comprise further processing apparatus apparatus 140 configured to form one or more products 145 from the one or more alcohols of alcohol product 135.

As noted above with reference to FIG. 1, method I includes, at step 10, collecting exhaust gas comprising carbon dioxide (CO₂) at a wellsite 111 to provide a collected exhaust gas 115. Collecting exhaust gas at 10 can be effected via exhaust gas collection apparatus 110 (FIG. 2). Exhaust gas collection apparatus 110 can include and/or obtain the collected exhaust gas 115 from field operating equipment 112 (FIG. 3) at a wellsite 111. The field operating equipment 112 can comprise one or more vehicles (e.g., diesel trucks, cars, etc.), pumps (e.g., hydraulic pumps, fracturing pumps, etc.), or other equipment at a wellsite 111 that produces an exhaust gas comprising CO₂ from which collected exhaust gas 115 is obtained. Exhaust gas collection apparatus 110 can further comprise piping configured to combine the exhaust gas from a plurality of the field operating equipment 112 and introduce it to CO₂ separation apparatus 120, storage apparatus to store the collected exhaust gas 115 prior to introduction into CO₂ separation apparatus 120, or a combination thereof. Collecting the collected exhaust gas 115 comprising CO₂ at 10 can be performed by piping exhaust gas from one or more pieces of field operating equipment or machinery 112 at a wellsite 111 to provide the collected exhaust gas 115.

The exhaust gas comprising CO₂ collected at step 10 can include greater than or equal to about 0.04, 0.1, 0.5, 1, 2, 3, 4, 5, 6, 7, 8, 9, 10, 20, 30, 40, 50, 60, 70, 80, 90, 95, or 100 volume percent (vol.%) CO₂. By way of examples, the collected exhaust gas comprising CO₂ 115 can include a waste gas, or one or more components thereof, produced at the wellsite 111 or another jobsite, such as, without limitation, one or more wellsites or industrial plants. The one or more industrial plants can include, without limitation, a cement plant, a chemical processing plant, a mechanical processing plant, a refinery, a steel plant, a power plant (e.g., a gas power plant, a coal power plant, etc.), or a combination and/or a plurality thereof. In embodiments, the exhaust gas comprising CO₂ 115 comprises a waste gas that is a product of fuel combustion, for example, the product of an internal combustion engine, or a gas fired turbine engine, such as, for example, from a microgrid having electric pumps. In embodiments, the internal combustion engine includes an engine fueled by diesel, natural gas, gasoline, or a combination thereof (e.g., a diesel engine, or a hybrid engine that is fueled by diesel and natural gas). The collected exhaust gas comprising CO₂ 115 can be produced at the wellsite 111 and/or another jobsite. A plurality of machinery 112 can be located and operated at a wellsite 111 for performing a subterranean formation operation, according to one or more embodiments of the present disclosure, and the collected exhaust gas comprising CO₂ 115 can, in embodiments, be obtained therefrom. For example, the exhaust gas comprising CO₂ from which the collected exhaust gas 115 can be produced at the wellsite 111 from machinery 112 used to perform a wellbore servicing operation. The machinery may include one or more internal combustion or other suitable engines that consume fuel to perform work at the wellsite 111 and produce exhaust gas comprising CO₂ from which collected exhaust gas 115 is collected.

The wellbore 101 at wellsite 111 may be a hydrocarbon-producing wellbore (e.g., oil, natural gas, and the like) or another type of wellbore for producing other resources (e.g., mineral exploration, mining, and the like). Machinery 112 typically associated with a subterranean formation operation related to a hydrocarbon producing wellbore, and from which the exhaust gas comprising CO₂ can be produced, can be utilized to perform such operations as, for example, a cementing operation, a fracturing operation, or other suitable operation where equipment is used to drill, complete, produce, enhance production, and/or work over the wellbore. Other surface operations may include, for example, operating or construction of a facility.

As depicted in FIG. 3, which is a schematic of a plurality of machinery 112 that may be located and operated a wellsite 111 for performing a subterranean formation operation and may produce exhaust gas comprising CO₂ from which collected exhaust gas 115 is collected, according to one or more embodiments of the present disclosure, the machinery 112 from which the exhaust gas comprising CO₂ can be produced, in embodiments, can include sand machinery 112A, gel machinery 112B, blender machinery 112C, pump machinery 112D, generator machinery 112E, positioning machinery 112F, control machinery 112G, and/or other machinery 112H. The machinery 112 may be, for example, truck, skid or rig-mounted, or otherwise present at the wellsite 111, without departing from the scope of the present disclosure. The sand machinery 112A may include transport trucks or other vehicles for hauling to and storing at the wellsite 111 sand for use in an operation. The gel machinery 112B may include transport trucks or other vehicles for hauling to and storing at the wellsite 111 materials used to make a gelled treatment fluid for use in an operation The blender machinery 112C may include blenders, or mixers, for blending materials at the wellsite 111 for an operation. The pump machinery 112D may include pump trucks or other vehicles or conveyance equipment for pumping materials down the wellbore 101 for an operation. The generator machinery 112E may include generator trucks or other vehicles or equipment for generating electric power at the wellsite 111 for an operation. The electric power may be used by sensors, control machinery, and other machinery. The positioning equipment 112F may include earth movers, cranes, rigs or other equipment to move, locate or position equipment or materials at the wellsite 111 or in the wellbore 101.

The control machinery 112G may include an instrument truck coupled to some, all, or substantially all of the other equipment at the wellsite 111 and/or to remote systems or equipment. The control machinery 112G may be connected by wireline or wirelessly to other equipment to receive data for or during an operation. The data may be received in real-time or otherwise. In another embodiment, data from or for equipment may be keyed into the control machinery.

The control machinery 112G may include a computer system for planning, monitoring, performing or analyzing the job. Such a computer system may be part of a distributed computing system with data sensed, collected, stored, processed and used from, at or by different equipment or locations. The other machinery 112H may include equipment also used at the wellsite 111 to perform an operation.

In other examples, the other machinery 112H may include personal or other vehicles used to transport workers to the wellsite 111 but not directly used at the wellsite 111 for performing an operation.

Many if not most of these various machinery 112 at the wellsite 11 accordingly utilize a diesel or other fuel types to perform their functionality. Such fuel is expended and exhausted as exhaust gas, such as exhaust gas including CO₂. The embodiments described herein provide a system and method for collecting, converting to one or more alcohols, and, thus, sequestering CO₂ from such machinery 112 located and operated at a wellsite 111, thus reducing atmospheric CO₂ emissions, while reducing material and time costs. It is to be appreciated that other configurations of the wellsite 111, including other machinery 112 at the wellsite 111 or another jobsite, may be employed, without departing from the scope of the present disclosure. Although a number of various machinery 112 at a jobsite (e.g., a wellsite 111) have been mentioned, many other machinery may utilize diesel or other fuel that creates exhaust gas including CO₂ that may conventionally be exhausted into the atmosphere, but herein utilized to form alcohol product or precursor 135 as described herein.

In some embodiments, the present disclosure provides capturing exhaust gas comprising CO₂ 115 from such machinery located and operated at a wellsite 111 and utilizing such exhaust gas to form alcohol product 135 as detailed herein.

Although described hereinabove with reference to a wellsite 111, the source of the collected exhaust gas comprising CO₂ 115 that is collected at step 10 of the method I can be any convenient CO₂ source. The CO₂ source can be a gaseous CO₂ source This gaseous CO₂ may vary widely, ranging from air, industrial waste streams, etc. As noted above, the gaseous CO₂ can, in certain instances, comprise an exhaust waste product from an industrial plant. The nature of the industrial plant may vary in these embodiments, where industrial plants of interest include power plants, chemical processing plants, and other industrial plants that produce exhaust gas comprising CO₂ as a byproduct. By waste stream is meant a stream of gas (or analogous stream) that is produced as a byproduct of an active process of the industrial plant, e.g., an exhaust gas. The gaseous stream may be substantially pure CO₂ or a multi-component gaseous stream that includes CO₂ and one or more additional gases Multi-component gaseous streams (containing CO₂) that may be employed as a CO₂ source in embodiments of the subject methods include both reducing, e.g., syngas, shifted syngas, natural gas (e.g., methane), and hydrogen and the like, and oxidizing condition streams, e.g., flue gases from combustion. Particular multi-component gaseous streams of interest that may be treated according to the subject invention include: oxygen containing combustion power plant flue gas, turbo charged boiler product gas, coal gasification product gas, shifted coal gasification product gas, anaerobic digester product gas, wellhead natural gas stream, reformed natural gas or methane hydrates, and the like.

As noted above, in embodiments, the collected exhaust gas comprising CO₂ 115 comprises greater than or equal to about 5, 6, 7, 8, 9, 10, 15, 20, 25, 30, 40, 50, 60, 70, 80, 90, 95, 96, 97, 98, 99, or 100 volume percent (vol%) CO₂. In embodiments, the exhaust gas comprising CO₂ 115 comprises primarily CO₂ (e.g., greater than or equal to about 50, 60, 70, 80, 90, 95, 96, 97, 98, 99, or 100 volume percent (vol%) CO₂). For example, when the exhaust gas comprising CO₂ 115 is obtained from a waste gas produced at a different jobsite (e.g., at an another jobsite) than the wellsite 111, CO₂ can be separated from the waste gas in order to reduce a volume of gas to be transported to the wellsite 111. For example, when the exhaust gas includes a flue gas from a power plant, which typically contains from about 7 to about 10 vol.% CO₂, the method I can further include transporting the exhaust gas comprising CO₂ (or a waste gas from which the exhaust gas comprising CO₂ 115 is obtained) from the another jobsite at which the waste gas is obtained to the wellsite 111. In embodiments, the method I can further include separating the exhaust gas from the waste gas comprising CO₂, to reduce a volume of gas, e.g., for transport. Although the separating of the exhaust gas comprising CO₂ from the waste gas can be performed at the wellsite 111 (e.g., after transport of the waste gas from the another jobsite at which the waste gas is obtained and/or produced to the wellsite 111), to facilitate transportation, the separating of the collected exhaust gas comprising CO₂ 115 from the waste gas can be performed at the another jobsite at which the waste gas is produced and/or obtained and, subsequently, the collected exhaust gas comprising CO₂ 115 can be transported to the wellsite 111.

As noted above, method I can comprise, at 20, separating CO₂ from the collected exhaust gas to provide a separated CO₂ 125. Separating the CO₂ from the collected exhaust gas 115 at 20 can comprise separating substantially pure CO₂ from the collected exhaust gas 115. That is, in embodiments, the separated CO₂ 125 is substantially pure CO₂. The substantially pure CO₂ (and the substantially pure separated CO₂ 125) can include greater than or equal to about 90, 95, 96, 97, 98, 99, 99.5, 99.8, 99.9, or 100 vol% CO₂. Separating CO₂ from the collected exhaust gas 115 to provide a separated CO₂ 125 at 20 can be effected by CO₂ separation apparatus 120. CO₂ separation apparatus 120 can comprise any apparatus operable to provide high purity (e.g., greater than or equal to 50, 60, 70, 80, 90, 95, 96, 97, 98, 98.5, 99, 99.5, 99.9, 99.99, or substantially 100 volume percent (vol%) CO₂ from the collected exhaust gas 115. In embodiments, CO₂ separation apparatus 120 can operate by separating via amine absorption, calcium oxide (CaO) absorption, filtration, packed bed, another technique, or a combination thereof. In embodiments, CO₂ separation apparatus 120 comprises batch reactor, continuous reactor, packed-bed column, fluidized pack column, or a combination thereof. In embodiments, the collected exhaust gas 115 collected at step 10 has a suitable concentration of CO₂ for production of the alcohol product 135 at step 30, and no separating step 20 or CO₂ separation apparatus 120 is utilized

As noted above, method I comprises, at 30, forming alcohol product or precursor 135 utilizing at least a portion of the separated CO₂ 125. The alcohol product 135 can be formed from the separated CO₂ using any known or yet to be discovered process. In embodiments, the forming of the alcohol product 135 at step 30 employs a catalyst or enzymatic conversion.

Traditional metal or organic based catalysts can be utilized for carbon capture and conversion. For this, a catalytic system can be utilized that involves the capture of CO₂ and alkali-metal hydroxides and alcohol as a formate ester facilitator. In such embodiments, useful molecules can be created on the fly by capturing carbon dioxide from the exhaust or flue gas and mixing it with hydrogen in the presence of metal catalyst. In embodiments, the catalyst can comprise copper, ruthenium, or zinc-based oxides, supported on alumina. The conversion of carbon dioxide into methanol can be depicted as in the following general equation, Eq. (1):

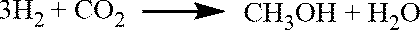

Without being limited by theory, the reaction sequence during the hydrogenation of the separated CO₂ 125 can be via a metal alkyl carbonate salt and the presence of metal, such as a ruthenium-based catalyst, to produce formate derivatives. The formate derivative(s) turn can be converted to alkyl formates and then to methanol as shown in Equation (2):

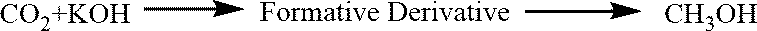

Alcoholic metal hydroxide can be used to form metal carbonate. In order to increase the boiling point and avoid toxicity, ethylene glycol can be utilized as a benign solution.

In embodiments, methane (e.g., as a byproduct of flareup) can be captured and utilized to make an alcohol product 135 comprising methane as a methanol precursor to the synthesis of methanol and/or ethanol, as shown in Equation (3):

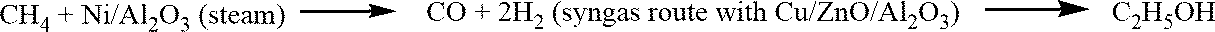

Accordingly, in embodiments, method I of continuously capturing CO₂ from the exhaust gas (e.g., of fracturing equipment) comprises: collecting exhaust gas at 10 and, at 30, providing alkali-metal hydroxides as a source for conversion of the CO₂ in the collected exhaust gas 115 to a methanol precursor or intermediate, such as a formate, utilizing hydrogen to push the reaction further to the final product methanol, and allowing the catalyst convert the formate to methanol. As described herein, the CO₂ can be collected from fracturing equipment during an operation at wellsite 111 and/or collected exhaust gas 115 or separated CO₂ 125 can be trucked in or brought in via a pipeline, etc from another wellsite 111 or from another jobsite, such as from a power plant, cement plant, etc.

Accordingly, with reference now to FIG. 4, in embodiments, the alcohol product 135 comprises a precursor to methanol comprising a formate derivative. In such embodiments, metal catalysts and alkali-metal hydroxides can be utilized to catalyze the conversion of CO₂ to formate derivatives, which can then be further converted to methanol. In such embodiments, forming the alcohol product 135 can comprise hydrogenating at least a portion of the separated CO₂ 125 in the presence of a catalyst to produce the formate derivative. In such embodiments, alcohol production apparatus 130 can include a formate production reactor 132, configured to produce a formate derivative 133 by hydrogenating (with hydrogen introduced as additional reactant 131) the at least the portion of the separated CO₂ 125 in the presence of a metal catalyst. The metal catalyst can comprise, for example, a ruthenium-based catalyst

The formate product 133 can be utilized and/or sold as-is, or can be converted at the wellsite 111 or another jobsite to methanol. For example, in embodiments, alcohol production apparatus 130 further includes a methanol production reactor 134 configured to convert the formate derivative or product 133 into methanol of alcohol product 135 (e.g., via alkyl formate, as described above). The methanol containing alcohol product 135 can be utilized or sold as-is, or, as detailed further herein, can be subjected to further processing at step 40 in further processing apparatus 140.

In embodiments, formation of the methanol in alcohol production apparatus 130 can be effected, for example, as described in J. Am. Chem. Soc. 2020, 142, 10, 4544-4549, entitled, “Hydroxide Based Integrated CO₂ Capture from Air and Conversion to Methanol,” and/or as described in Fuel Processing Technology, May 2016, 145, 42-61, entitled, “Synthesis of Methanol from Methane: Challenges and Advances on the Multi-Step (Syngas) and One-Step Routes (DMTM)”.

In embodiments, alcohol product 135 comprises ethanol. In some such embodiments, forming the alcohol product at step 30 comprises forming ethanol by reacting the at least the portion of the separated CO₂ 125 with water (e.g., additional reactant 131 comprises water) in the presence of an electrocatalyst to produce ethanol. Reacting the at least the portion of the separated CO₂ 125 with the water 131 in the presence of the electrocatalyst can further comprise contacting the electrocatalyst with the at least the portion of the separated CO₂ 125 and applying a voltage thereto to convert the CO₂ in the at least the portion of the separated CO₂ 125 into ethanol of alcohol product 135. The voltage can be about -1.2 volts, in embodiments. The reacting the at least the portion of the separated CO₂ 125 with the water 131 at step 30 can be effected at about ambient temperature (e.g., a temperature in a range of from about 66° F. to about 70° F. (from about 18.9° C. to about 21.1° C.), from about 67° F. to about 71° F. (from about 19.4° C. to about 21.7° C.), from about 67.5° F. to about 70.5° F. (from about 19.7° C. to about 21.4 °Cor less than or equal to about 90° F., 85° F., 80° F., 75° F., 70° F., or 65° F. (less than or equal to about 32.2° C., 29.4° C., 26.7° C., 23.98° C., 21.1° C., or 18.3° C.)), and/or about atmospheric pressure (e.g., a pressure in a range of from about 0.9 atmospheres (atm) to about 1.1 atm (from about 91.2 kPa to about 111.4 kPa), from about 0.95 atm to about 1.05 atm (from about 96.3 kPa to about 106.4 kPa), from about 0.8 atm to about 1.2 atm (from about 81.0 kPa to about 121.6 kPa), or less than or equal to about 1.5, 1.4, 1.3, 1.2, 1.1, or 1.0 atm (less than or equal to about 152.0, 141.8, 131.7, 121.6, 111.4, or 101.3 kPa)).

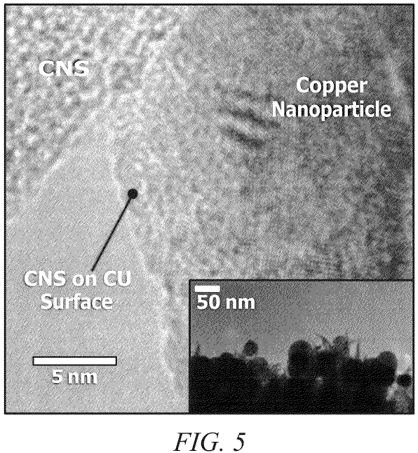

As depicted in FIG. 5, which is a high-resolution transmission electron microscopy of an electrocatalyst 136 suitable for use in the system of FIG. 2, according to embodiments of this disclosure. Electrocatalyst 136 comprises electrodeposited copper nanoparticles on a carbon nanospike (CNS) electrode. The electrocatalyst 136 utilized to produce ethanol can thus, in embodiments, comprise carbon nanospikes and copper nanoparticles, with the copper nanoparticles supported on and/or embedded in the CNS. As seen in FIG. 5, the electrodeposited copper nanoparticles are imbedded in N-doped CNS providing intimate contact between copper surface and alpha-carbon reactive sites. In embodiments, the carbon nanospikes are doped with nitrogen, comprise layers of puckered carbon, and/or comprise a curled tip. In embodiments, the electrocatalyst comprises, for example, an electrocatalyst substantially as described in U.S. Pat. App. No. 15/143,651, entitled, “Electrochemical Catalyst for Conversion of CO₂ to Ethanol,” the disclosure of which is hereby incorporated herein for purposes not contrary to this disclosure.

In such embodiments, alcohol production apparatus 130 can comprise an electrocatalytic reactor comprising electrocatalyst 136. The electrocatalytic reactor is configured for the reduction of CO₂ in the at least the portion of the separated CO₂ 125 to ethanol, for example, as shown in Equation (4):

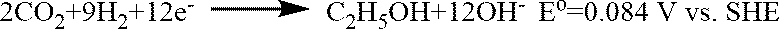

The alcohol product 135 (e.g., alcohol product comprising methanol, ethanol, a precursor thereof, or a combination thereof) can be utilized onsite at wellsite 111 or at another jobsite, sold, or otherwise utilized to benefit. Alternatively or additionally, as noted above, method I can further comprise further processing the alcohol product 135 at 40, as described further hereinbelow.

Further processing the alcohol product 135 at step 40 can comprise, for example, removing one or more component 141 from the alcohol product 135 and/or converting the one or more alcohol of alcohol product 135, optionally in the presence of one or more additional reactant(s) 142, into another product 145. For example, removing one or more component 141 from the alcohol product 135 can include removing one or more contaminant, one or more non-alcohol component (or non-desired alcohol component), water, or a combination thereof from alcohol product 135. Accordingly, as depicted in FIG. 4, a system 100 of this disclosure can comprise a further processing apparatus 140 configured to separate one or more components 141 from the alcohol product 135 and/or convert the one or more alcohols in alcohol product 135 to another product (e.g., via reaction with one or more reactants 142), thus providing product stream 145.

By way of example, as noted hereinabove, alcohol product 135 can comprise methanol. Methanol can be utilized to produce methyl amines, methyl halides, methyl ethers, methyl esters, and etc. In embodiments, the methanol of alcohol product 135 can be utilized as a precursor to other commodity chemical(s), including, without limitation, formaldehyde, acetic acid, methyl tert-butyl ether (MTBE), methyl benzoate, anisole, peroxyacids, or another chemical. In such embodiments, further processing apparatus 140 can comprise one or more reactors configured to convert the methanol in alcohol product 135 to a product 145 including one or more of methyl amines, methyl halides, methyl ethers, methyl esters, or another product produced from methanol. For example, the methanol can be utilized as a precursor to other commodity chemicals, such as, without limitation, formaldehyde, acetic acid, methyl tert-butyl ether (MTBE), methyl benzoate, anisole, peroxyacids, etc.

By way of specific example, in embodiments, methanol of alcohol product 135 can be utilized in conjunction with isobutene (additional reactant 141) to produce MTBE as product 145. The MTBE can be sold for use as an octane booster in gasoline or utilized in the production of polymers, for example.

By way of further example, in embodiments, the methanol of alcohol product 135 can be utilized to produce acetic acid as product 145, for example, via the Cativa process,

In embodiments, the methanol of alcohol product 135 can be utilized in gas to liquids processes, such as the conversion of methanol to hydrocarbons (MtH), the conversion of methanol to gasoline (MtG), and/or the conversion of methanol to propylene (MtP). Such conversions can be catalyzed by zeolites as heterogeneous catalysts. Accordingly, the product 145 can include hydrocarbons, propylene, gasoline, or another liquid produced from the methanol of alcohol product 135.

In embodiments, at least a portion of the methanol of alcohol product 135 can be utilized along with a co-solvent as a component (e.g., 3%) of gasoline at the wellsite 111 or another jobsite.

As noted hereinabove, in embodiments, alcohol product 135 comprises ethanol. Accordingly, the herein disclosed system and method enable collection of exhaust gas comprising CO₂ (at step 10), separation (as needed) of the CO₂ in the collected exhaust gas (at step 20), and utilization of the separated CO₂ 125 (e.g., at step 30) to produce liquid fuel (e.g., ethanol). The ethanol of alcohol product 135 can be utilized at the wellsite 111 or another jobsite (e.g., another wellsite) as a fuel for various apparatus, such as vehicles. Alternatively, in embodiments, the liquid fuel can be sold to advantage.

As depicted in FIG. 2, in embodiments, a system of this disclosure, such as system 100 of FIG. 2 (e.g., a system 100 including a formate production reactor 132 and/or a methanol production reactor 134, as depicted in the embodiment of FIG. 4), or one or more components thereof (e.g., exhaust gas collection apparatus 110, CO₂ separation apparatus 120, alcohol production apparatus 130, further processing apparatus 140, or a combination of one or more components thereof) can be provided on one or more skids 150 (e.g., a trailer skid), whereby at least a portion the separated CO₂ 125 can be converted to alcohol product 135 comprising one or more alcohols, precursors thereof, or a combination thereof at the wellsite 111. For example, in embodiments, such as depicted in FIG. 2, CO₂ separation apparatus 120, alcohol production apparatus 130, and/or further processing apparatus 140 can be located on one or more skids 150, in embodiments.

Also provided herein is a method comprising: producing methanol, ethanol, a precursor thereof, or a combination thereof utilizing, as a reactant, carbon dioxide separated from exhaust gas produced at a wellsite. Producing the methanol, ethanol, precursor thereof, or combination thereof and/or a separating of the carbon dioxide (e.g., from which the methanol, ethanol, precursor thereof, or combination thereof is produced) from the exhaust gas to provide the separated exhaust gas, or both can be performed substantially continuously or intermittently. Producing the methanol, ethanol, precursor thereof, or combination thereof and/or separating of the carbon dioxide (e.g., from which the methanol, ethanol, precursor thereof, or combination thereof is produced) from the exhaust gas to provide the separated exhaust gas, or both can be performed at a same jobsite (e.g., wellsite 111) or at different jobsites (e.g., the producing the methanol, ethanol, precursor thereof, or combination thereof can be effected at a first jobsite (e.g., a wellsite 111) and/or the separating of the carbon dioxide (e.g., from which the methanol, ethanol, precursor thereof, or combination thereof is produced) from the exhaust gas to provide the separated exhaust gas can be performed at another jobsite (e.g., another wellsite 111).

The system and method of this disclosure can provide for continuous, semi-continuous, or intermittent collecting of exhaust gas 115 from field operating equipment 112 at a wellsite 111 and utilization of the collected exhaust gas 115 to produce alcohol product 135. The alcohol product 135 can be utilized to benefit at the wellsite 111, for example, as a liquid fuel, and/or can be sold for profit (e.g., at a location remote from wellsite 111).

Rather than or in addition to injecting downhole collected exhaust gas 115 comprising CO₂ and/or separated CO₂ 125 separated from collected exhaust gas 115 for sequestration purposes, the CO₂ in the exhaust gas 115 and/or the separated CO₂ 125 obtained therefrom can be converted by the system and method of this disclosure to convert the CO₂, at wellsite 111 (or another jobsite or a different wellsite from which the exhaust gas was collected), to a useful alcohol product 135 comprising one or more alcohols, such as methanol, ethanol, a precursor of ethanol, a precursor of methanol, or a combination thereof.

In embodiments, the system and method described herein provide for capturing CO₂ from a large or effectively unlimited amount of exhaust gas produced at a wellsite 111, and utilizing the reaction mechanism through catalytic conversion to produce useful chemicals. In embodiments, the conversion of the separated CO₂ to alcohol product 135 and/or further product 145 is effected at low ambient operating temperature to moderately high temperatures (eg., without requiring a heat exchanger) and/or atmospheric pressure.

In embodiments, the system of this disclosure (or one or more components thereof) can be provided on a skid 150 (e.g., a trailer skid), whereby at least a portion the CO₂ in the exhaust gas 115 and/or the separated CO₂ 125 obtained therefrom can be converted to alcohol product 135 comprising one or more alcohols at the wellsite 111.

In embodiments, at least a portion of the system 100 (e.g., alcohol production apparatus 130, further processing apparatus 140) is provided as a small-scale alcohol production plant (e.g., on one or more skids 150) at wellsite 111, whereby alcohol product 135 can be produced on location, one or more product 145 can be produced from the one or more alcohols of alcohol product 135 on location, and/or a the alcohol product 135 or the product 145 produced therefrom can be utilized at the wellsite 111 or another jobsite/wellsite and/or sold for profit.

### ADDITIONAL DISCLOSURE

The following are non-limiting, specific embodiments in accordance with the present disclosure:

In a first embodiment, a method comprises: collecting exhaust gas comprising carbon dioxide (CO₂) at a wellsite to provide a collected exhaust gas; separating CO₂ from the collected exhaust gas to provide a separated CO₂; and forming an alcohol product utilizing at least a portion of the separated CO₂, wherein the alcohol product comprises methanol, ethanol, a precursor thereof, or a combination thereof.

A second embodiment can include the method of the first embodiment, wherein the alcohol product comprises a precursor to methanol comprising a formate derivative, and wherein forming the alcohol product further comprises hydrogenating at least a portion of the separated CO₂ in the presence of a catalyst to produce the formate derivative, and converting the formate derivative to alkyl formate, and to methanol.

A third embodiment can include the method of the first embodiment, wherein the alcohol product comprises ethanol, and wherein forming the alcohol product comprises forming ethanol by reacting the at least the portion of the separated CO₂ with water in the presence of an electrocatalyst to produce ethanol.

A fourth embodiment can include the method of the third embodiment, wherein reacting the at least the portion of the separated CO₂ with the water in the presence of the electrocatalyst further comprises contacting the electrocatalyst with the at least the portion of the separated CO₂ and applying a voltage thereto to convert the CO₂ in the at least the portion of the separated CO₂ into ethanol.

A fifth embodiment can include the method of the fourth embodiment, wherein the voltage is about -1.2 volts.

A sixth embodiment can include the method of any one of the fourth or the fifth embodiments, wherein to the reacting the at least the portion of the separated CO₂ with the water is effected at about ambient temperature (e.g., a temperature in a range of from about 66° F. to about 70° F. (from about 18.9° C. to about 21.1° C.), from about 67° F. to about 71° F. (from about 19.4° C. to about 21.7° C.), from about 67.5° F. to about 70.5° F. (from about 19.7° C. to about 21.4° C.), or less than or equal to about 90° F., 85° F., 80° F., 75° F., 70° F., or 65° F. (less than or equal to about 32.2° C., 29.4° C., 26.7° C., 23.98° C., 21.1° C., or 18.3° C.)), and/or about atmospheric pressure (e.g., a pressure in a range of from about 0.9 atmospheres (atm) to about 1.1 atm (from about 91.2 kPa to about 111.4 kPa), from about 0.95 atm to about 1.05 atm (from about 96.3 kPa to about 106.4 kPa), from about 0.8 atm to about 1.2 atm (from about 81.0 kPa to about 121.6 kPa), or less than or equal to about 1.5, 1.4, 1.3, 1.2, 1.1, or 1.0 atm (less than or equal to about 152.0, 141.8, 131.7, 121.6, 111.4, or 101.3 kPa)).

A seventh embodiment can include the method of any one of the third to sixth embodiments, wherein the electrocatalyst comprises carbon nanospikes and copper nanoparticles, wherein the copper nanoparticles are supported on and/or embedded in the carbon nanospikes.

An eighth embodiment can include the method of the seventh embodiment, wherein the carbon nanospikes are doped with nitrogen.

A ninth embodiment can include the method of the eighth embodiment, wherein the carbon nanospikes comprise layers of puckered carbon.

A tenth embodiment can include the method of the ninth embodiment, wherein the carbon nanospikes comprise a curled tip.

In an eleventh embodiment, a system comprises: an exhaust gas collection system configured for collecting exhaust gas comprising carbon dioxide (CO₂) at a wellsite to provide a collected exhaust gas; a CO₂ separation apparatus configured for separating CO₂ from the collected exhaust gas to provide a separated CO₂; and an alcohol production apparatus configured for forming an alcohol product utilizing at least a portion of the separated CO₂, wherein the alcohol product comprises methanol, ethanol, a precursor thereof, or a combination or thereof.

A twelfth embodiment can include the system of the eleventh embodiment, wherein the alcohol product comprises a methanol precursor, and wherein the alcohol production apparatus comprises a formate production reactor configured to produce a formate derivative by hydrogenating the at least the portion of the separated CO₂ in the presence of a metal catalyst.

A thirteenth embodiment can include the system of the twelfth embodiment further comprising a methanol formation reactor configured to convert the formate derivative to methanol.

A fourteenth embodiment can include the system of any one of the twelfth or thirteenth embodiments, wherein the metal catalyst comprises a ruthenium-based catalyst.

A fifteenth embodiment can include the system of any one of the eleventh to fourteenth embodiments, wherein the alcohol production apparatus comprises an electrocatalytic reactor comprising an electrocatalyst, and wherein the electrocatalytic reactor is configured for the reduction of CO₂ in the at least the portion of the separated CO₂ to ethanol.

A sixteenth embodiment can include the system of the fifteenth embodiment, wherein the electrocatalyst comprises carbon nanospikes and copper nanoparticles, wherein the copper nanoparticles are supported on and/or embedded in the carbon nanospikes

A seventeenth embodiment can include the system of the sixteenth embodiment, wherein the carbon nanospikes are doped with nitrogen.

An eighteenth embodiment can include the system of the sixteenth embodiment, wherein the carbon nanospikes comprise layers of puckered carbon

A nineteenth embodiment can include the system of any one of the sixteenth to eighteenth embodiments, wherein the carbon nanospikes comprise a curled tip.

A twentieth embodiment can include the system of any one of the sixteenth to nineteenth embodiments, wherein to the electrocatalytic reactor is operable at about ambient temperature (e.g., a temperature in a range of from about 66° F. to about 70° F. (from about 18.9° C. to about 21.1° C.), from about 67° F. to about 71° F. (from about 19.4° C. to about 21.7° C.), from about 67.5° F. to about 70.5° F. (from about 19.7° C. to about 21.4°Cor less than or equal to about 90° F., 85° F., 80° F., 75° F., 70° F., or 65° F. (less than or equal to about 32.2° C., 29.4° C., 26.7° C., 23.98° C., 21.1° C., or 18.3° C.)), and/or about atmospheric pressure (e.g., a pressure in a range of from about 0.9 atmospheres (atm) to about 1.1 atm (from about 91.2 kPa to about 111.4 kPa), from about 0.95 atm to about 1.05 atm (from about 96.3 kPa to about 1064 kPa), from about 0.8 atm to about 12 atm (from about 81.0 kPa to about 121.6 kPa), or less than or equal to about 1.5, 1.4, 1.3, 1.2, 1.1, or 1.0 atm (less than or equal to about 152.0, 141.8, 131.7, 121.6, 111.4, or 101.3 kPa)).

In a twenty first embodiment, a method comprises producing methanol, ethanol, a precursor thereof, or a combination thereof utilizing, as a reactant, carbon dioxide separated from exhaust gas produced at a wellsite.

A twenty second embodiment can include the method of the twenty first embodiment, wherein the producing, a separating of the carbon dioxide from the exhaust gas to provide the separated exhaust gas, or both are performed substantially continuously or intermittently.

While embodiments have been shown and described, modifications thereof can be made by one skilled in the art without departing from the spirit and teachings of this disclosure. The embodiments described herein are exemplary only, and are not intended to be limiting. Many variations and modifications of the embodiments disclosed herein are possible and are within the scope of this disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R1, and an upper limit, Ru, is disclosed, any number falling within the range is specifically disclosed. In particular, the following numbers within the range are specifically disclosed: R=R1 +k^{∗} (Ru-Rl), wherein k is a variable ranging from 1 percent to 100 percent with a 1 percent increment, i.e., k is 1 percent, 2 percent, 3 percent, 4 percent, 5 percent, ..... 50 percent, 51 percent, 52 percent, ....., 95 percent, 96 percent, 97 percent, 98 percent, 99 percent, or 100 percent. Moreover, any numerical range defined by two R numbers as defined in the above is also specifically disclosed. Use of broader terms such as comprises, includes, having, etc. should be understood to provide support for narrower terms such as consisting of, consisting essentially of, comprised substantially of, etc. When a feature is described as “optional,” both embodiments with this feature and embodiments without this feature are disclosed. Similarly, the present disclosure contemplates embodiments where this “optional” feature is required and embodiments where this feature is specifically excluded.

Accordingly, the scope of protection is not limited by the description set out above but is only limited by the claims which follow, that scope including all equivalents of the subject matter of the claims. Each and every claim is incorporated into the specification as embodiments of the present disclosure. Thus, the claims are a further description and are an addition to the embodiments of the present disclosure. The discussion of a reference herein is not an admission that it is prior art, especially any reference that can have a publication date after the priority date of this application. The disclosures of all patents, patent applications, and publications cited herein are hereby incorporated by reference, to the extent that they provide exemplary, procedural, or other details supplementary to those set forth herein.

What is claimed is:

1. A method comprising:
collecting exhaust gas comprising carbon dioxide (CO₂) at a wellsite to provide a collected exhaust gas;
separating CO₂ from the collected exhaust gas to provide a separated CO₂; and
forming an alcohol product utilizing at least a portion of the separated CO₂, wherein the alcohol product comprises methanol, ethanol, a precursor thereof, or a combination thereof.

2. The method of claim 1, wherein the alcohol product comprises a precursor to methanol comprising a formate derivative, and wherein forming the alcohol product further comprises hydrogenating at least a portion of the separated CO₂ in the presence of a catalyst to produce the formate derivative, and converting the formate derivative to alkyl formate, and to methanol.

3. The method of claim 1, wherein the alcohol product comprises ethanol, and wherein forming the alcohol product comprises forming ethanol by reacting the at least the portion of the separated CO₂ with water in the presence of an electrocatalyst to produce ethanol.

4. The method of claim 3, wherein reacting the at least the portion of the separated CO₂ with the water in the presence of the electrocatalyst further comprises contacting the electrocatalyst with the at least the portion of the separated CO₂ and applying a voltage thereto to convert the CO₂ in the at least the portion of the separated CO₂ into ethanol.

5. The method of claim 4, wherein the voltage is about -1.2 volts.

6. The method of claim 4, wherein to the reacting the at least the portion of the separated CO₂ with the water is effected at about ambient temperature, and/or about atmospheric pressure.

7. The method of claim 3, wherein the electrocatalyst comprises carbon nanospikes and copper nanoparticles, wherein the copper nanoparticles are supported on and/or embedded in the carbon nanospikes.

8. The method of claim 7, wherein the carbon nanospikes are doped with nitrogen.

9. The method of claim 8, wherein the carbon nanospikes comprise layers of puckered carbon.

10. The method of claim 9, wherein the carbon nanospikes comprise a curled tip.

11. A system comprising:
an exhaust gas collection system configured for collecting exhaust gas comprising carbon dioxide (CO₂) at a wellsite to provide a collected exhaust gas;
a CO₂ separation apparatus configured for separating CO₂ from the collected exhaust gas to provide a separated CO₂; and
an alcohol production apparatus configured for forming an alcohol product utilizing at least a portion of the separated CO₂, wherein the alcohol product comprises methanol, ethanol, a precursor thereof, or a combination or thereof.

12. The system of claim 11, wherein the alcohol product comprises a methanol precursor, and wherein the alcohol production apparatus comprises a formate production reactor configured to produce a formate derivative by hydrogenating the at least the portion of the separated CO₂ in the presence of a metal catalyst.

13. The system of claim 12, wherein the system further comprises a methanol formation reactor configured to convert the formate derivative to methanol.

14. The system of claim 12, wherein the metal catalyst comprises a ruthenium-based catalyst.

15. The system of claim 11, wherein the alcohol production apparatus comprises an electrocatalytic reactor comprising an electrocatalyst, and wherein the electrocatalytic reactor is configured for the reduction of CO₂ in the at least the portion of the separated CO₂ to ethanol.

16. The system of claim 15, wherein the electrocatalyst comprises carbon nanospikes and copper nanoparticles, wherein the copper nanoparticles are supported on and/or embedded in the carbon nanospikes.

17. The system of claim 16, wherein the carbon nanospikes are doped with nitrogen, comprise layers of puckered carbon, and/or comprise a curled tip.

18. The system of claim 16, wherein to the electrocatalytic reactor is operable at about ambient temperature, and/or about atmospheric pressure.

19. A method comprising:
producing methanol, ethanol, a precursor thereof, or a combination thereof utilizing, as a reactant, carbon dioxide separated from exhaust gas produced at a wellsite.

20. The method of claim 19, wherein the producing, a separating of the carbon dioxide from the exhaust gas to provide the separated exhaust gas, or both are performed substantially continuously or intermittently.
